(19) United States
(12) Patent Application Publication
(10) Pub. No.: US 2022/0346940 A1
(43) Pub. Date: Nov. 3, 2022

(54) A MEDICAL SUPPORT DEVICE

(71) Applicant: Dr Hedén AB, Stockholm (SE)

(72) Inventor: Per HEDÉN, Stockholm (SE)

(21) Appl. No.: 17/618,113

(22) PCT Filed: Jun. 9, 2020
(86) PCT No.: PCT/SE2020/050579

§ 371 Date: Dec. 10, 2021

(30) Foreign Application Priority Data
Jun. 13, 2019 (SE) 1950712-8

(51) Int. Cl. A61F 2/12

(52) U.S. Cl. CPC A61F 2/12; A61L 2430/04; A61F 2220/0008

(57) ABSTRACT

A medical support device (1), for use in a woman's breast as an implant without incapsulated material, wherein the medical support device (1) comprises at least a wall shaped portion (2) having a first side (3), which shows a bulge out of a plane (4), and a second side (5) of the wall shaped portion (2) has a corresponding cavity, for example a dome shape, wherein the medical support device (1) comprises an elastomer. The device will keep endogenous tissue and/or fat and/or added material, such as hyaluronic acid, in place inside of deep gland tissue of a woman's breast by means of being attached to tissue, such as muscle. A method of producing and to use such a medical support device (1).

The present invention concerns a medical support device for use in a human body part, a method of producing it and to use it.

### BACKGROUND

Previously are implants filled with a filling, such as silicone gel or saline, known. There is always a risk of leakage of the filling or encapsulating of the implant.

It is also known to use a matrix to support an implant within a human body part, the matrix may be absorbable or not by the human body.

### SUMMARY OF THE INVENTION

The aim of the present invention is to be able to support and/or augment a human body part without the previously known drawbacks.

According to a first aspect of the invention a medical support device, for use in a human body part is provided. The device comprises at least a wall shaped portion having a first side, which shows a bulge out of a plane, and a second side of the wall shaped portion has a corresponding cavity, for example as a dome shape, wherein the medical support device comprises an elastomer. The wall shaped portion is curved around more than one axis or has a draped shape. The medical support device is open from the plane into the cavity, i.e. never closed such as, for example, a common breast implant filled with liquid silicone gel or saline.

With the medical support device according to the invention it will be possible to keep endogenous tissue and/or fat and/or added material, such as hyaluronic acid in place in the human body part. An advantage with this solution is that it will be permeable for x-ray radiation when filled with endogenous tissue and/or fat, which is important for secure mammography.

According to an embodiment the wall thickness, i.e. between the first side and second side, is more or less the same over the wall shaped portion. Preferably, the medical support is pre-shaped.

According to an embodiment a rim in the plane is at least partially present along an edge of the cavity. The rim will make it easier to apply sutures to the medical support device when fastening it to the tissue of the body part. Attaching the medical support device will in turn lead to less problems with malpositioning, such as rotation, and lateral and bottoming out movement of the implant. It will also make sure the material in the cavity of the medical support device stays in place.

According to an embodiment the device is made of an elastomer and provided with a matrix at least at the rim.

According to an embodiment the device is made of an elastomer and provided with a matrix at least partially at the second side, cavity side, of the wall shaped portion.

According to an embodiment device is used in a breast, preferably in a woman's breast.

According to a second aspect of the invention a method of producing a medical support device provided, whereby a mandrel having a desired shape is dipped in an unhardened elastomer to shape the device. Preferably at least a piece of matrix is arranged on the mandrel before or on the device after the dipping.

According to a third aspect of the invention a method of using such a medical support device according is provided. An implant without incapsulated material is formed, where the medical support device keeps endogenous tissue and/or fat and/or added material, such as hyaluronic acid, in place in the human body part.

### SHORT DESCRIPTION OF DRAWINGS

The present invention will now be described in more detail under referral to the enclosed drawings, in which:

FIGS. 1a, b and c show an embodiment of the invention in a cross-sectional view, a plan view and a side view, respectively.

FIGS. 2a, b, c and d show different embodiments having a rim in a cross-sectional view and two plan views, respectively.

FIG. 3a-g show different embodiments of the invention in cross-sectional views.

FIGS. 4a and b show embodiments of different shapes from the side.

FIGS. 5a and b show embodiments with different shapes in a plan view.

FIGS. 6a, b and c show different embodiments of mandrels for the production of embodiments of the invention.

FIG. 7 shows an example of a use of the invention.

FIG. 8 shows an example of a use of the invention.

### DETAILED DESCRIPTION OF EMBODIMENTS OF THE INVENTION

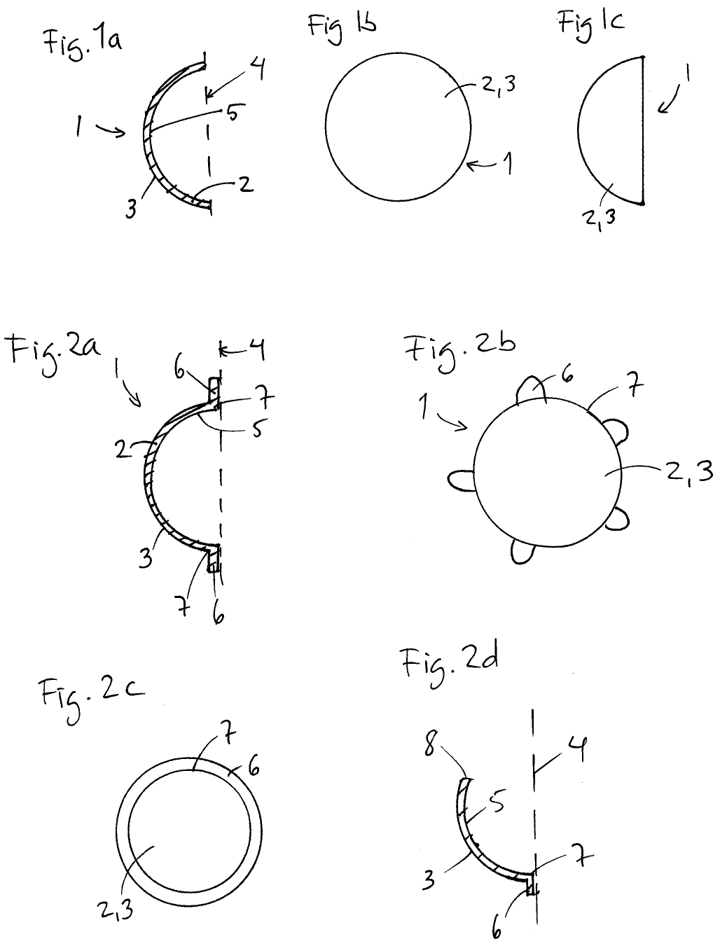

In FIGS. 1a, b and c an embodiment of a medical support device 1 of the present invention is shown. The medical support device 1 comprises at least a wall shaped portion 2. It has a first side 3, which shows a bulge out of a plane 4 and a second side 5 that has a corresponding cavity. For example, the wall shaped portion 2 may have a dome shape, although it is possible to have any particular shape according to the purpose of the supporting in the human body.

The wall shaped portion 2 is curved over at least two different thought axes or draped, i.e. it does not have shape of a partial cylinder. Preferably it has a thickness between the first side 3 and the second side 5 which is more or less the same over the bulge/cavity. In FIG. 1b the medical support device 1 is shown in a plan view from the first side 2. In the shown embodiment the device has a more or less circular footprint, although any shape would be possible. In FIG. 1c the embodiment of FIGS. 1a and b is shown from the side.

The medical support device 1 is for supporting endogenous tissue and/or fat in place in a human body part. Any suitable body part may receive the medical support device 1, such as in the buttocks or breasts, although it would probably be mostly used in women's breasts. The medical device 1 will support endogenous tissue or fat or both against gravitational pull. It is also conceivable to add other materials suitable to be in a human body, such as hyaluronic acid. Or only use other material or materials in the cavity portion of the medical support device 1.

The medical support device 1 comprises either an elastomer or matrix, or both. The elastomer material may be of any kind suitable to be used with a human body and being pliable. The elastomer may for example be a silicon rubber, preferably of the type used in breast implants. The matrix may be built up by biological or synthetic materials or a combination thereof. It could comprise absorbable and/or non-absorbable material. For example, it could be an acellular dermal matrix.

In FIG. 2a an embodiment of a medical support device 1 is shown in cross section where at least a portion of a rim 6 is provided at an edge 7 of the cavity or bulge in the plane 4. The at least one portion of the rim 6 may be more easily attached to tissue in a human body than attaching only an edge 7. Preferably, any suture may be used for this, absorbable or not.

In FIG. 2b an embodiment is shown in a plan view where several portions of the rim 6 is provided and in FIG. 2c a full rim 6 surrounding the edge 7 is shown. In FIG. 2d another embodiment is shown where the wall shaped portion 2 is cut off 8 in its upper portion and having a rim 6 around the edge 7 left in the plane 4 so that a pocket is formed when attached to tissue in the human body. All the shown embodiment in FIG. 2 may comprise an elastomer and/or matrix.

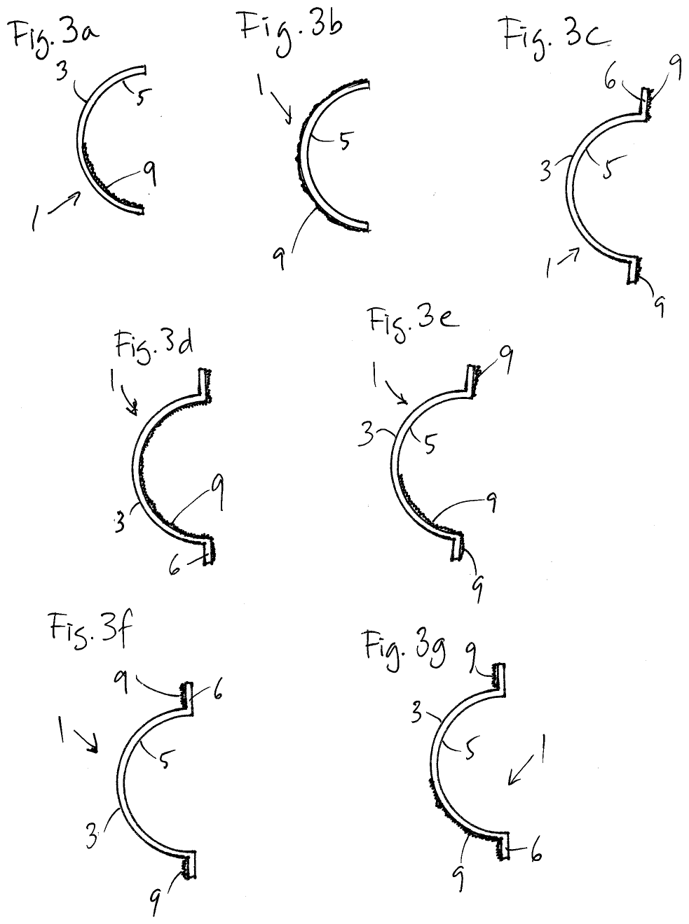

Different embodiments of how a combination of an elastomer and matrix will now be exemplified in FIGS. 3a-g. In FIG. 3a a dome-shaped medical support device 1 is shown made of elastomer and having a portion of matrix 9 provided on the second side 5 in the lower region. Obviously, matrix 9 may be provided all over the second side 5 or, as shown in FIG. 3b, all over the first side 3, or over a portion of the first side 3. In FIG. 3c a medical support device 1 having a rim 6 is shown where matrix 9 is provided at the rim 6 on the same side as the second side 5 of the wall shaped portion 2. It is also conceivable to have matrix 9 on the other side, as is shown in FIG. 3f or on both sides. In FIG. 3d matrix 9 is provided both in the cavity, the second side 5, and at the rim 6. In FIGS. 3e and 3g matrix 9 is provided on a lower portion of the cavity and bulge, respectively, as well as along the rim 6.

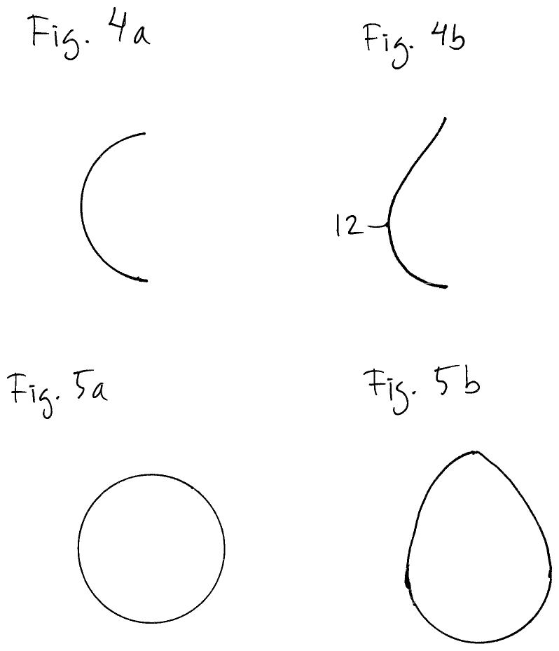

Obviously, the shape of the medical support device 1 may differ according to its use and purpose. In FIG. 4a a more or less dome-shape is shown, preferably a partial sphere, and in FIG. 4b an anatomical shape is shown. The projection may differ, too, as well as the height of a projection point 12. The footprint of the medical support device 1 may also differ according to its use and purpose. In FIGS. 5a and 5b two different examples are shown, circular having the same height as width; or having different height compared to the width, respectively. The height may be longer than the width or vice versa. For example, the footprint may be oval or as shown in FIG. 5b, drop shaped.

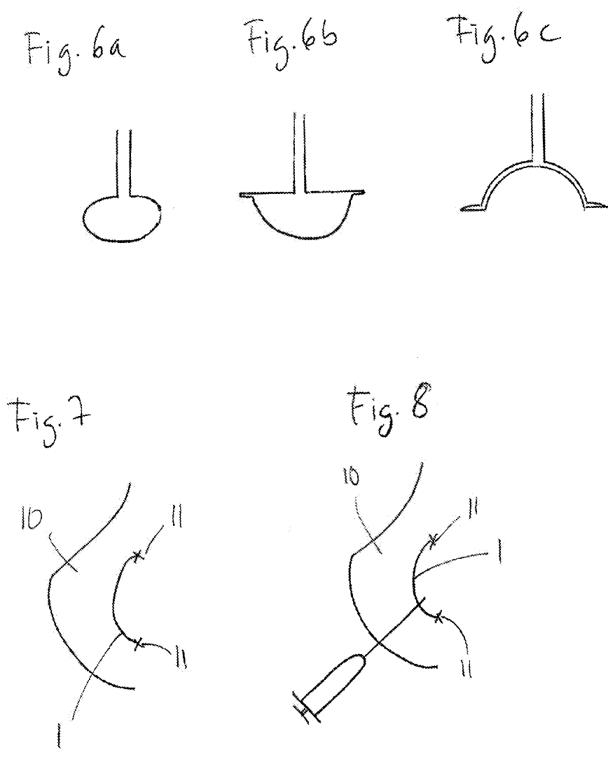

Now examples of how a medical support device 1 may be produced will be described. In FIGS. 6a, b and c three different mandrels are shown. The mandrel is dipped in an elastomer in a flowing condition. Then it is taken up and the elastomer hardens into a pliable condition. This could be effectuated by means of heat or added substances, for example. The dipping usually is repeated a desired number of times. The elastomer is taken off the mandrel, and if needed cut to desired shape. It is possible to turn it inside out, i.e. the surface of the elastomer closest to the mandrel will instead become the bulge side, the first side 3.

In case a special surface is desired, such as a textured surface, it is possible to provide it on the first side 3 or second side 5, respectively, or on both sides. Different roughness from fine to coarse may be provided by having a surface structure on the mandrel. It is also possible to imprint a texture on the outermost layer, before hardening. For example, it is possible to use polyurethane or a salt to imprint.

If a rim 6 is desired a mandrel according to FIGS. 6b and 6c may be used. If a combination of elastomer and matrix is desired parts of matrix may be arranged at the mandrel at desired positions, or over the full mandrel surface, before the dipping. Alternatively, the matrix is arranged after the dipping before hardening.

The medical support device 1 may be provided in a range of sizes making it possible to find a suitable size for the special needs of each patient.

In FIG. 7 a use of the medical support device 1 is shown. The medical support device 1 is positioned inside a human body part 10, in this case a female breast. In the present case the deep gland tissue and/or fat is separated and a portion of it is provided inside of the cavity, i.e. on the second side 5 of the medical support device 1. The medical support device is attached with sutures 11. The sutures are preferably of the absorbable type. The medical support device may for example be attached in a muscle, the breast muscle in the shown case. It is also conceivable to provide fat or other tissue, preferably endogenous, within the cavity of the medical support device in addition to gland tissue or only fat or only other tissue. It is also conceivable to add hyaluronic acid or other medical injectable material in the cavity, alone or mixed with endogenous tissue of any or several kinds.

In FIG. 8 it is shown the possibility to add, for example hyaluronic acid or fat, by means of a syringe into a cavity of a medical support device already in place in a human body part 10. This puncturing will not do any harm as no harmful material may leak out as it would with a common breast implant filled with silicone gel.

1. A medical support device, for use in a woman's breast as an implant without encapsulated material, wherein the device comprises at least a wall shaped portion having a first side, which shows a bulge out of a plane, and a second side of the wall shaped portion has a cavity corresponding to the bulge, for example as a dome shape, the medical support device comprises an elastomer, the support device is arranged to support endogenous tissue and/or endogenous fat in place by being attached to tissue, such as muscle, the cavity of the support device is fillable with endogenous tissue and the support device is positionable inside of deep gland tissue of the woman's breast and a portion of the deep gland tissue and/or fat is thus provided inside the cavity of the support device.

2. The medical support device according to claim 1, wherein a matrix is comprised in the medical support device.

3. The medical support device according to claim 1, wherein a rim in the plane is at least partially present along an edge of the cavity of the wall shaped portion.

4. The medical support device according to claim 3, wherein the device is made of an elastomer and provided with a matrix at least at the rim.

5. A method of producing a medical support device according to claim 1, whereby a mandrel having a desired shape is dipped in an unhardened elastomer one or several times and hardened in order to shape the medical support device.

6. The method according to claim 5, whereby at least a piece of matrix is arranged on the mandrel or on the device after the dipping.

7. A method of using a medical support device in a woman's breast as an implant without encapsulated material, wherein the medical support device comprises at least a wall shaped portion having a first side, which shows a bulge out of a plane, and a second side of the wall shaped portion has a cavity corresponding to the bulge, for example as a dome shape, the medical support device comprises an elastomer, in such a manner that the support device will support endogenous tissue and/or endogenous fat in place by being attached to tissue, such as muscle, so that the cavity of the support device is filled with endogenous tissue and the support device is positioned inside of deep gland tissue of the woman's breast and a portion of the deep gland tissue and/or fat will thus be provided inside the cavity of the support device.
